(19) United States
(12) Patent Application Publication
(10) Pub. No.: US 2025/0327402 A1
(43) Pub. Date: Oct. 23, 2025

(54) NATURAL METAL ENRICHMENT USING PRODUCED HYDROCARBON FIELD WATERS

(71) Applicant: Saudi Arabian Oil Company, Dhahran (SA)

(72) Inventor: Simon A. Stewart, Dhahran (SA)

(21) Appl. No.: 18/642,978

(22) Filed: Apr. 23, 2024

(51) Int. Cl. E21B 43/34; E21B 43/40; E21B 49/08

(52) U.S. Cl. CPC E21B 49/086; C02F 1/14; E21B 43/40; G01N 33/24

(57) ABSTRACT

To naturally retrieve metals in metal-rich produced water obtained from hydrocarbon reservoirs, a subterranean zone that is a source of metal-rich produced water is identified. An evaporation area is identified. The evaporation area is at a location that is geographically optimal to the subterranean zone. The evaporation area is rich in deposits of evaporites that resulted from evaporation of water that accumulated in the evaporation area. The metal-rich produced water is obtained from the subterranean zone. A portion of the metal-rich produced water is flowed to the evaporation area. Evaporation of the portion of the metal-rich produced water causes metal in the portion of the metal-rich produced water to be deposited in the evaporation area. At least a portion of the metal deposited in the evaporation area is retrieved.

### TECHNICAL FIELD

This disclosure describes recovering metal present in produced waters obtained from hydrocarbon fields (i.e., a region with one or more hydrocarbon reservoirs) using evaporative techniques.

### BACKGROUND

Semi-arid, arid or hyper-arid climate near coastal regions results in the formation of mudflats or sandflats in which evaporites accumulate. Evaporites are water-soluble sedimentary mineral deposits that result from concentration and crystallization by evaporation of an aqueous solution. In regions experiencing arid climates (e.g., hot, sunny locations such as the desert), evaporites accumulate in natural evaporation areas. In some geographical regions, such evaporation areas are called sabkhas or salars. Such evaporation areas are filled with mineral-bearing waters from underlying groundwater sources. Due to the arid climate, evaporation rates in such locations can be extremely high. Mineral-bearing waters, which may accumulate in such evaporation areas, naturally evaporate at higher rates (compared to areas in other regions). Produced waters obtained from hydrocarbon fields is another example of mineral-bearing waters that can contain high concentrations of mineral deposits.

### SUMMARY

This disclosure describes techniques relating to natural metal enrichment using produced hydrocarbon field waters. In some implementations described in this disclosure, produced waters from hydrocarbon fields can be routed to evaporation areas formed in arid climates. In such areas, the produced water can evaporate leaving dissolved metals in the areas. In certain geomorphologies, this technique can take advantage of existing naturally-enclosed ponds such as intermontane basins or interdune sabkhas, which provide a natural evaporating pond system that can be harnessed.

The details of one or more implementations of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a schematic diagram showing an example of an integrated producing hydrocarbon field and a water evaporation area.

FIG. 2 is a flowchart of an example of a process of evaluating options for metal enrichment projects.

FIG. 3 is a flowchart of an example of a process of retrieving metals from produced water.

Like reference numbers and designations in the various drawings indicate like elements.

### DETAILED DESCRIPTION

Hydrocarbon fields are large swaths of land with hydrocarbons (e.g., oil, gas, combination of them) entrapped in subsurface reservoirs. The process of retrieving the entrapped hydrocarbons is called hydrocarbon production. Reservoirs have an operating life during which hydrocarbon can be produced. For operating hydrocarbon fields (particularly, oil fields) with multiple such reservoirs, water is produced along with the hydrocarbons. For example, in the final phases of the life of an oil field, the percentage of water to the total fluids produced from the oil field (also known as water cut) can reach over 90%. Often, some or all of the produced water is recirculated back into the hydrocarbon field to maintain reservoir pressure and sustain continued production. Doing so also avoids needing to dispose the produced water by other means.

Produced water from hydrocarbon fields are often saline and called brine. The concentration of salts of sodium, chloride, magnesium, potassium and calcium in produced water is high (up to 30% by volume of brine). Sometimes, produced water can also contain appreciable amounts of metals like chromium, cobalt, copper, nickel, silver, zinc, tungsten and lithium, as well as nonmetallic elements such as bromine and iodine. Such metals can be used in other applications, e.g., energy transition applications. This disclosure describes techniques to identify produced water that is rich in such valuable metals and to retrieve the metals from the produced water in a cost effective manner.

Such retrieval is implemented by routing the metal-rich produced water to evaporation areas that naturally occur in hot, sunny regions or any region that experiences arid climate. In such regions, the rates of mineral concentration in evaporation areas are high. Moreover, such ponds tend to be dry demonstrating excess evaporative capacity relative to that required to fully evaporate the fluids received by natural recharge processes. The metal-rich produced water naturally evaporates in the evaporation areas leaving behind metal deposits. The metal deposits can then be retrieved and further processed to obtain metals that can be used in various applications. Utilization of natural evaporating locations is established art. For example, the Salar de Atacama is a natural evaporating location where Lithium-rich brine is pumped from shallow wells into partitioned ponds within an intermontane basin. Solar energy concentrates the brine until Lithium-rich sludge is pumped or trucked to refining infrastructure where the Lithium concentration is raised to commercial grade. Costs are involved in the initial recovery of Lithium-rich brine and the construction of partitioned brine ponds.

Implementing the techniques described here can provide the following advantages. Valuable metals contained in produced water can be retrieved using techniques that are not only less resource intensive but also more environmentally friendly. Obtaining the produced water leverages techniques that are already being deployed to produce hydrocarbons. Therefore, such techniques do not consume significantly additional resources such as new water-producing wells. Further, evaporation areas often occur naturally and do not need to be separately constructed. Moreover, blending evaporites from the produced water with evaporites that have formed and/or accumulated already in the evaporation areas adds to the volume of metals that can be retrieved. In addition, the produced water serves as an additional recharge mechanism to the evaporation area. In doing so, the produced water harnesses and boosts an existing natural process. In sum, utilizing the evaporation potential of natural evaporation areas in hot climates to evaporate metal-rich produced water from oil fields can support sustainability and other applications, mitigating the capital expenditure and minimizing the environmental impact that are key challenges in energy transition projects.

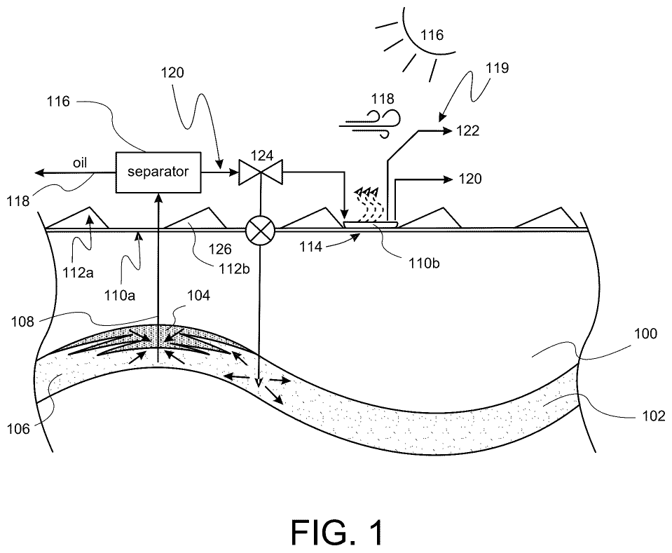

FIG. 1 is a schematic diagram showing an example of an integrated producing hydrocarbon field and a water evaporation area. A subterranean zone 100 (e.g., a formation, a portion of a formation or multiple formations). One or more hydrocarbon reservoirs (e.g., a hydrocarbon reservoir 102) can be found in the subterranean zone 100. A hydrocarbon reservoir is a layer of porous rock that is generally filled with brine, with oil locally ponded where subsurface conditions are suitable. Hydrocarbons 104 are entrapped in the subterranean zone 100. The hydrocarbons can be produced from the hydrocarbon fields by forming one or more wellbores (e.g., the wellbore 108) from the surface to the hydrocarbon reservoir through the subterranean zone. Over the life of the hydrocarbon reservoir 102, the quantity of water 106 in the reservoir increases. For example, the water may be present in regions below the hydrocarbon-bearing formations and rise into the hydrocarbon-bearing formations over time to replace the hydrocarbons that have been removed by production. In such instances, the produced hydrocarbon fluids 108 (i.e., the fluids that are raised to the surface) include a mixture of the hydrocarbons 104 (e.g., oil) and the water 106 (called produced water). In some instances, the produced water can be rich in (i.e., have high concentrations of) valuable metals such as chromium, copper, nickel, silver and lithium. As an example, brines containing more than 100 parts per million (0.01%) can be considered a basis for Lithium concentration and extraction

In some implementations, the producing hydrocarbon field, particularly, one from which metal-rich produced water is obtained, can be integrated with evaporation areas (e.g., an evaporation area 110a, an evaporation area 110b). The pre-requisites for such integration include climate that is hot and arid (or hyper-arid) such that daily evaporation rates are high. Desert regions are examples of such regions. The pre-requisites also include operating hydrocarbon fields in which water with high concentrations of dissolved metals are found. The subterranean zone 100 is an example of such an operating hydrocarbon field. The pre-requisites further include hydrogeological and geomorphological setting that includes evaporation areas, e.g., natural evaporation areas. In particular, evaporation areas that are recharged by (i.e., periodically filled by) shallow groundwater brines 114 (e.g., the evaporation area 110b) are ideal for the integration with the producing hydrocarbon field because the containment architecture and evaporation processes are already in place and need only to be harnessed or amended to handle the additional load of brines produced from deep reservoirs in the oilfield. In hot, sunny regions (e.g., desert regions), such evaporation areas can be formed between sand dunes (e.g., sand dunes 112a and 110b), such pond areas may be called sabkhas in local geographic schemes.

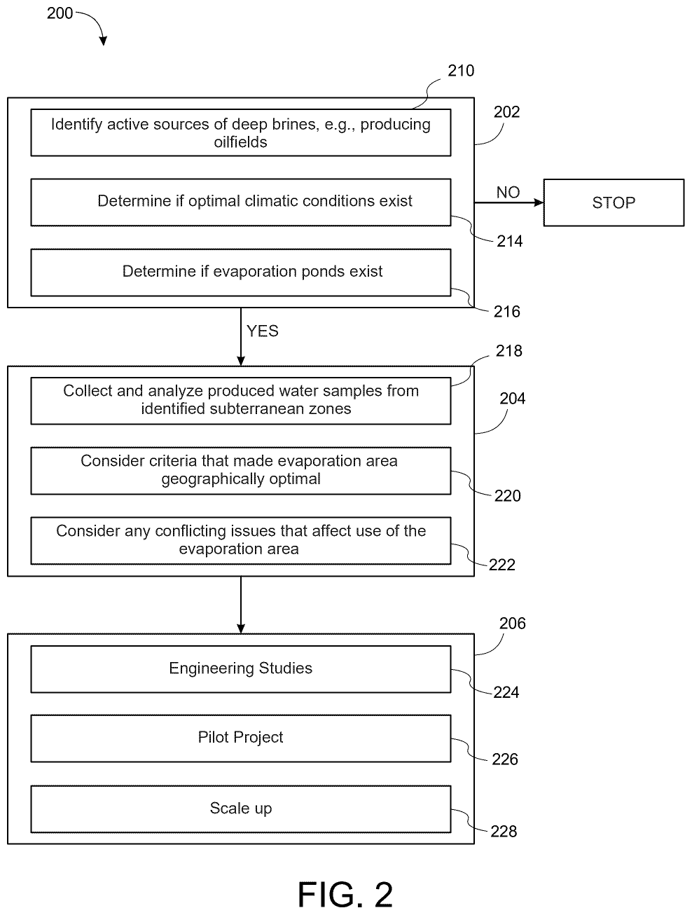

FIG. 2 is a flowchart of an example of a process 200 of evaluating options for metal enrichment projects. Evaluating options includes identifying hydrocarbon fields that produce metal-rich water and identifying evaporation areas that are geographically proximal to such hydrocarbon fields. Having identified such hydrocarbon fields and evaporation areas, metals in the produced water can be recovered/retrieved by integrating the processes to obtain produced water with evaporation in the identified evaporation areas.

With the pre-requisites satisfied, the process 200 can be implemented in three steps-a regional assessment step 202, a portfolio development step 204 and an implementation step 206.

The regional assessment step 202 includes multiple steps. At 210, active sources of deep brines are identified. The active sources can include hydrocarbon fields such as the subterranean zone 100. More specifically, the active sources can include hydrocarbon reservoirs (e.g., the hydrocarbon reservoir 102) in each hydrocarbon oilfield. An active source is one from which hydrocarbons are being produced and in which the water cut is high (i.e., greater than a threshold water cut). Variables such as total produced water volume and metal concentration also determine if a source is an active source. The brines produced from deep-oil-bearing reservoirs can include metal-rich produced water with high concentration of valuable metals. At 216, a determination is made if optimal climatic conditions exist in a region near the active sources identified at 210. As explained above, optimal climatic conditions include hot, sunny conditions, arid/hyper-arid conditions, conditions that facilitate natural evaporation (e.g., high winds) or any combination of them. At 214, a determination is made if evaporation areas exist in the regions identified in 212. If the outcome of the determination steps at 212 and 214 is negative (decision branch “NO”), then the process may need to be terminated as being unfeasible. On the other hand, if the outcome of the determination steps at 212 and 214 is positive (decision branch “YES”), then the process 200 proceeds to the portfolio development step 204.

The portfolio development step 204 also includes multiple steps. At 218, produced water samples can be collected from each of the hydrocarbon fields identified at step 210 of the regional assessment step 202. For example, the produced water samples can be collected from one or more or all of the hydrocarbon reservoirs (e.g., the hydrocarbon reservoir 102) in the identified hydrocarbon fields (e.g., the subterranean zone 100) from which hydrocarbons and water are produced. For example, when hydrocarbon fluids 108 are produced from one or more wellbores and flowed through flowlines, a sample trap can be fluidically coupled to the flowlines to obtain samples of the hydrocarbon fluids. The hydrocarbons and water can be separated to collect the produced water sample. Alternatively, the produced hydrocarbon fluids 108 can be flowed through a water-oil separator (WOSEP) 116 that can separate the hydrocarbons and water (e.g., by gravimetric separation). In some implementations, the water and hydrocarbons can be separated at a Gas Oil Separation Plant (GOSP). The oil stream 118 and the produced water stream 120 can be flowed away from each other, and a produced water sample can be drawn from the produced water stream 120. In these manners, produced water samples can be collected from one or more or all hydrocarbon reservoirs in the hydrocarbon field.

The collected produced water samples are then analyzed to determine concentration of one or more valuable metals in the samples. An example method of determining the elemental content of brine is inductively coupled plasma-optical emission spectroscopy (ICP-OES). Other methods include atomic absorption.

In some implementations, the subterranean zone 100 can include multiple hydrocarbon reservoirs, each of which produces water along with hydrocarbons. In such implementations, one or more samples can be collected from each hydrocarbon reservoir and analyzed for metal concentrations. The results of the analysis can be ranked, e.g., from samples with the most to least metal concentrations. Based on the ranking of the samples, the hydrocarbon reservoirs can be ranked as sources of produced water with most to least metal concentrations. The rankings can be used to identify an optimal hydrocarbon reservoir from which to obtain metal-rich produced water. Water samples can also be taken from non-hydrocarbon bearing areas to generally characterize the spatial distribution of metals and minerals in the reservoir brines of the region or sedimentary basin. This regional approach allows for ranking of oil fields even if water samples from a given oilfield are unavailable, or if that oil field has yet to reach a stage in production maturity when there is appreciable watercut.

Returning to the portfolio development step 204, criteria that make the evaporation area identified (in steps 212 and 214 of the regional assessment step 202) are considered at step 220. For example, considering the criteria includes evaluating if each identified evaporation area is geographically optimal (for example, nearby) to an identified hydrocarbon oilfield. One criterion is that the evaporation areas are rich in deposits of evaporites that resulted from evaporation of water that naturally accumulated in the evaporation area. This indicates that the environment is suitable for the evaporation and mineral concentration process. For example, the evaporation area 110b can be identified. Evaporite deposition in the evaporation area 110b could have been due to the flow of shallow groundwater brines 114 followed by natural evaporation due to hot, sunny (and sometimes windy) weather conditions. A hot, arid region can have multiple evaporation areas. However, not all may be geographically optimal for integration with a hydrocarbon field to retrieve metals from produced water. For example, the evaporation area 110b can be geographically optimal because it nearest to the subterranean zone 100 compared to other evaporation areas. The evaporation area 110b can also be geographically optimal because the weather conditions at the evaporation area improve and/or aid in natural evaporation compared to weather conditions at other evaporation areas. A larger evaporation area would be more optimal compared to a smaller one. Another optimal condition would be proximity of the evaporation area to civil infrastructure such as a large road or railroad. Evidence that the evaporation area experiences groundwater recharge is another optimal condition.

In some implementations, evaluating an evaporation area can include obtaining a spatial distribution of hydrocarbon fields and a geomorphology map that includes geographic locations of evaporation areas. The spatial distribution of hydrocarbon fields is then overlaid over the geomorphology map. Based on the overlaying, the evaporation area (e.g., the evaporation area 110b) that is nearest to the hydrocarbon field (e.g., the subterranean zone 100) is identified. Spatial information such as locations of oilfields, evaporating ponds or anything else is routinely captured in Geographical Information Systems (GIS) within which any number of layers can be concurrently displayed. Such layers can contain point data (e.g. wells), lines (e.g. roads or pipelines) or areas (oil fields or evaporation ponds), or any other kind of information. The information itself can be stored and utilized in the form of images (e.g. raster files) or precise data (e.g. vector files). The process of layering and interpreting such information can be thought of as applying criteria such as site selection criteria. The results of the layering can be interpreted manually (e.g. visually/intuitively) or could be codified for computer determination and ranking.

At 222, any conflicting issues that affect use of the evaporation area are considered. For example, an overlay of all existing infrastructure or planned infrastructure is added on top of the overlay described above. Doing so identifies evaporation areas whose use may conflict with the utilization of the evaporation area for the purposes described in this disclosure. Such evaporation areas can be excluded because existing or planned infrastructure would interfere with flowing produced water from the identified oilfields to such ponds. In this manner, the portfolio development step 204 is implemented to identify suitable hydrocarbon oilfields that produce metal-rich produced water and suitable evaporation areas to which the produced water can be flowed. Natural evaporation in the evaporation areas allows recovering or retrieving the valuable metals in such produced water.

In the implementation step 206, engineering processes are implemented. At 224, engineering studies are conducted to develop design plans and equipment to obtain produced water from the hydrocarbon fields and to flow the obtained produced water to the evaporation areas. Such equipment can include flow equipment including flowlines, pumps, valves and associated flow equipment using which produced water from the hydrocarbon reservoir can be flowed to an evaporation area. A goal of the engineering studies is to minimize energy consumption and implement sustainable practices that conserve resources as needed and that are also environmentally friendly. Having conducted the engineering studies, a pilot project is deployed at step 226. In the pilot project, produced water from a hydrocarbon reservoir is flowed to an evaporation area.

In some implementations, not all of the produced water is flowed to an evaporation area. Instead, the produced water is split into two streams, e.g., at a valve 124 (FIG. 1). One of the split streams is flowed to evaporation area. The other split stream of produced water is pumped, e.g., using a pump 126 (FIG. 1) back into the hydrocarbon reservoir. The returning of produced water to an oil-producing reservoir generally has a beneficial effect on oil production due to subsurface pressure maintenance, as well as disposing of the brine which may not be chemically similar to existing near-surface waters.

After the water evaporates due to heat from the sun 116 (FIG. 1) and, in some instances, due to wind 118 (FIG. 1), the deposits 119 (FIG. 1) left behind in the evaporation area are retrieved. Such deposits (e.g., concentrated liquids) can include salt, which can be processed in salt processing plants 120 (FIG. 1). Metal in the deposits can be separated in metal separation plants 122 (FIG. 1). Separation can be implemented, e.g., by precipitation, ion exchange, solvent extraction and similar metal separation techniques. At 228 and after successfully demonstrating the pilot project (step 226), the process can be scaled up to flow industrial scale volumes of metal-rich produced water obtained from multiple hydrocarbon reservoirs in multiple oilfields to multiple evaporation areas, each determined to be geographically optimal to at least one hydrocarbon reservoir.

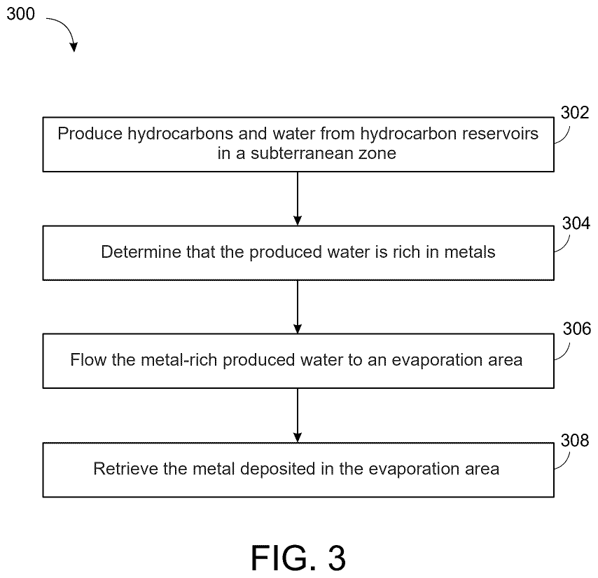

FIG. 3 is a flowchart of an example of a process 300 of retrieving metals from produced water. At 302, hydrocarbons and water are produced from hydrocarbon reservoirs in hydrocarbon fields. For example, wells are formed in hydrocarbon fields (e.g., the subterranean zone 100) to produce hydrocarbons entrapped in one or more hydrocarbon reservoirs (e.g., the hydrocarbon reservoir 102). Towards the end of the production life of a well, water is produced with the hydrocarbons.

At 304, it is determined that the produced water is rich in metals. For example, produced water can be tested to determine a concentration of one or more metals (e.g., the metals mentioned above). Upon determining that a concentration of a metal in the produced water is greater than a threshold concentration, then that produced water is designated as produced water rich in that metal. Determining the concentration and comparing with a threshold can reveal that produced water streams obtained from different hydrocarbon reservoirs are rich in different metals. As described above, such determination can be implemented by sampling produced water streams and analyzing the samples using techniques mentioned above.

At 306, the metal-rich produced water is flowed to an evaporation area. The evaporation area is one that previously has been determined to be geographically optimal to the hydrocarbon field from which the produced water is obtained. Before flowing the metal-rich produced water to the evaporation area, the produced water and hydrocarbons are separated. To flow the metal-rich produced water, flow equipment (e.g., flowlines, pumps, valves and other flow equipment) can be deployed between the hydrocarbon reservoirs from which the hydrocarbons and water are produced and the evaporation areas. Natural evaporation at the evaporation area causes the metals to be deposited on the evaporation area.

At 308, at least a portion of the metal deposited in the evaporation area can be retrieved. For example, natural evaporation at the evaporation area can result in deposition of salts in the produced water in addition to the metals. To retrieve the metals, the salts and metals are first separated. Then, the metal deposits can be processed and retrieved at metal separation plants.

### EXAMPLES

Certain aspects of the subject matter described here can be implemented as a method. A subterranean zone that is a source of metal-rich produced water is identified. An evaporation area is identified. The evaporation area is at a location that is geographically optimal to the subterranean zone. The evaporation area is rich in deposits of evaporites that resulted from evaporation of water that accumulated in the evaporation area. The metal-rich produced water is obtained from the subterranean zone. A portion of the metal-rich produced water is flowed to the evaporation area. Evaporation of the portion of the metal-rich produced water causes metal in the portion of the metal-rich produced water to be deposited in the evaporation area. At least a portion of the metal deposited in the evaporation area is retrieved.

An aspect combinable with any other aspect includes the following features. To identify the subterranean zone that is a source of metal-rich produced water, produced water samples are collected from the subterranean zone. The produced water samples are analyzed to determine concentration of metals in the collected produced water samples.

An aspect combinable with any other aspect includes the following features. The subterranean zone includes multiple hydrocarbon reservoirs from which produced water is obtained. To collect the produced water samples from the subterranean zone, a produced water sample is collected from each hydrocarbon reservoir. The produced water sample from each hydrocarbon reservoir is analyzed to determine concentration of metals in each produced water sample collected from each hydrocarbon reservoir.

An aspect combinable with any other aspect includes the following features. Based on results of analyzing the produced water sample from each hydrocarbon reservoir, the multiple hydrocarbon reservoirs are ranked based on concentrations of metals in produced water collected from each hydrocarbon reservoir of the multiple hydrocarbon reservoirs.

An aspect combinable with any other aspect includes the following features. The evaporation area that is nearest to the subterranean zone is geographically optimal to the subterranean zone. To identify the evaporation area that is nearest to the subterranean zone, a spatial distribution of subterranean zones is overlaid over a map that includes geographic locations of evaporation areas. Based on the overlaying, the evaporation area that is nearest to the subterranean zone is identified.

An aspect combinable with any other aspect includes the following features. To identify the evaporation area that is nearest to the subterranean zone based on the overlaying, one or more evaporation areas are excluded based on existing or planned infrastructure that interferes with flowing produced water to the one or more evaporation areas.

An aspect combinable with any other aspect includes the following features. To obtain the metal-rich produced water from the subterranean zone, hydrocarbons are produced from one or more hydrocarbon reservoirs in the subterranean zone. The metal-rich produced water is produced with the produced hydrocarbons. The metal-rich produced water is separated from the produced hydrocarbons.

An aspect combinable with any other aspect includes the following features. After separating the metal-rich produced water from the produced hydrocarbons, the metal-rich produced water is split into the first portion and a remaining portion. The remaining portion is injected into the one or more hydrocarbons from which the produced hydrocarbons were produced.

Certain aspects of the subject matter described here can be implemented as a method. Hydrocarbons are produced from one or more hydrocarbon reservoirs in a subterranean zone. Produced water is produced with the produced hydrocarbons. It is determined that the produced water is rich in metals. The metal-rich produced water is flowed to an evaporation area. Evaporation of the portion of the metal-rich produced water causes metal in the portion of the metal-rich produced water to be deposited in the evaporation area. At least a portion of the metal deposited in the evaporation area is retrieved.

An aspect combinable with any other aspect includes the following features. The produced water is separated from the produced hydrocarbons before flowing the metal-rich produced water to the evaporation area.

An aspect combinable with any other aspect includes the following features. To determine that the produced water is rich in metals, a produced water sample is collected from the one or more hydrocarbon reservoirs. The produced water sample is analyzed to determine concentration of metals in the produced water sample.

An aspect combinable with any other aspect includes the following features. Before flowing the metal-rich produced water to the evaporation area, it is determined that the evaporation area is geographically optimal to the one or more hydrocarbon reservoirs.

An aspect combinable with any other aspect includes the following features. To flow the metal-rich produced water to the evaporation area, the metal-rich produced water is split into the first portion and a remaining portion. The first portion is flowed into the evaporation area. The remaining portion is injected into the one or more hydrocarbon reservoirs from which the produced hydrocarbons are produced.

An aspect combinable with any other aspect includes the following features. To split the metal-rich produced water into the first portion and the remaining portion, the metal-rich produced water is received via a flowline at a three-way valve. The three-way valve splits the metal-rich produced water into the first portion and the remaining portion.

An aspect combinable with any other aspect includes the following features. To inject the remaining portion into the one or more hydrocarbon reservoirs, the remaining portion is pumped into the one or more hydrocarbon reservoirs.

Thus, particular implementations of the subject matter have been described. Other implementations are within the scope of the following claims.

1. A method comprising:
identifying a subterranean zone that is a source of metal-rich produced water;
identifying an evaporation area that is at a location that is geographically optimal to the subterranean zone, wherein the evaporation area is rich in deposits of evaporites that resulted from evaporation of water that accumulated in the evaporation area;
obtaining the metal-rich produced water from the subterranean zone;
flowing a portion of the metal-rich produced water to the evaporation area, wherein evaporation of the portion of the metal-rich produced water causes metal in the portion of the metal-rich produced water to be deposited in the evaporation area; and
retrieving at least a portion of the metal deposited in the evaporation area.

2. The method of claim 1, wherein identifying the subterranean zone that is a source of metal-rich produced water comprises:
collecting produced water samples from the subterranean zone; and
analyzing the produced water samples to determine concentration of metals in the collected produced water samples.

3. The method of claim 2, wherein the subterranean zone comprises a plurality of hydrocarbon reservoirs from which produced water is obtained, and wherein collecting the produced water samples from the subterranean zones comprises:
collecting a produced water sample from each hydrocarbon reservoir; and
analyzing the produced water sample from each hydrocarbon reservoir to determine concentration of metals in each produced water sample collected from each hydrocarbon reservoir.

4. The method of claim 3, further comprising, based on results of analyzing the produced water sample from each hydrocarbon reservoir, ranking the plurality of hydrocarbon reservoirs based on concentrations of metals in produced water collected from each hydrocarbon reservoir of the plurality of hydrocarbon reservoirs.

5. The method of claim 1, wherein the evaporation area that is nearest to the subterranean zone is geographically optimal to the subterranean zone, wherein identifying the evaporation area that is nearest to the subterranean zone comprises:
overlaying a spatial distribution of subterranean zones over a map that includes geographic locations of evaporation areas; and
identifying, based on the overlaying, the evaporation area that is nearest to the subterranean zone.

6. The method of claim 5, wherein identifying the evaporation area that is nearest to the subterranean zone based on the overlaying comprises excluding one or more evaporation areas based on existing or planned infrastructure that interferes with flowing produced water to the one or more evaporation areas.

7. The method of claim 1, wherein obtaining the metal-rich produced water from the subterranean zone comprises:
producing hydrocarbons from one or more hydrocarbon reservoirs in the subterranean zone, wherein the metal-rich produced water is produced with the produced hydrocarbons; and
separating the metal-rich produced water from the produced hydrocarbons.

8. The method of claim 7, further comprising, after separating the metal-rich produced water from the produced hydrocarbons:
splitting the metal-rich produced water into the first portion and a remaining portion; and
injecting the remaining portion into the one or more hydrocarbons from which the produced hydrocarbons were produced.

9. A method comprising:
producing hydrocarbons from one or more hydrocarbon reservoirs in a subterranean zone, wherein produced water is produced with the produced hydrocarbons;
determining that the produced water is rich in metals;
flowing the metal-rich produced water to an evaporation area, wherein evaporation of the portion of the metal-rich produced water causes metal in the portion of the metal-rich produced water to be deposited in the evaporation area; and
retrieving at least a portion of the metal deposited in the evaporation area.

10. The method of claim 9, further comprising separating the produced water from the produced hydrocarbons before flowing the metal-rich produced water to the evaporation area.

11. The method of claim 9, wherein determining that the produced water is rich in metals comprises:
collecting a produced water sample from the one or more hydrocarbon reservoirs; and
analyzing the produced water sample to determine concentration of metals in the produced water sample.

12. The method of claim 9, further comprising, before flowing the metal-rich produced water to the evaporation area, determining that the evaporation area is geographically optimal to the one or more hydrocarbon reservoirs.

13. The method of claim 9, wherein flowing the metal-rich produced water to the evaporation area comprises:
splitting the metal-rich produced water into the first portion and a remaining portion;
flowing the first portion into the evaporation area; and
injecting the remaining portion into the one or more hydrocarbon reservoirs from which the produced hydrocarbons were produced.

14. The method of claim 13, wherein splitting the metal-rich produced water into the first portion and the remaining portion comprises:
receiving the metal-rich produced water via a flowline at a three-way valve; and
splitting, by the three-way valve, the metal-rich produced water into the first portion and the remaining portion.

15. The method of claim 13, wherein injecting the remaining portion into the one or more hydrocarbon reservoirs comprises pumping the remaining portion into the one or more hydrocarbon reservoirs.
